(19) United States
(12) Patent Application Publication
(10) Pub. No.: US 2015/0196367 A1
(43) Pub. Date: Jul. 16, 2015

(54) MEDICAL DEVICE

(71) Applicant: Siemens Aktiengesellschaft, Munich (DE)

(72) Inventors: Hans-Jürgen Müller, Pretzfeld (DE); Andreas Wiesinger, Erlangen (DE)

(73) Assignee: Siemens Aktiengesellschaft, Munich (DE)

(21) Appl. No.: 14/421,197

(22) PCT Filed: Jul. 19, 2013
(86) PCT No.: PCT/EP2013/065315

§ 371 Date: Feb. 12, 2015

(30) Foreign Application Priority Data
Aug. 24, 2012 (DE) 102012215130.3

(51) Int. Cl. A61B 19/00; A61B 5/055

(52) U.S. Cl. CPC A61B 19/5202; A61B 5/055; A61B 2019/5206; A61B 2562/164; A61B 2019/521

(57) ABSTRACT

An embodiment of the invention relates to a medical device with a tubular interior space for accommodating the subject to be examined, a cladding of the tubular interior space facing towards the subject to be examined and a device for lighting the tubular interior space, which includes at least one lamp. The lamp is mounted on the side of the cladding of the tubular interior space facing towards the subject to be examined and forms the tunnel contour of the tubular interior space.

### PRIORITY STATEMENT

This application is the national phase under 35 U.S.C. §371 of PCT International Application No. PCT/EP2013/065315 which has an International filing date of Jul. 19, 2013, which designated the United States of America, and which claims priority to German patent application DE 102012215130.3 filed Aug. 24, 2012, the entire contents of each of which are hereby incorporated herein by reference.

### FIELD

At least one embodiment of the invention generally relates to a medical device having a tubular interior space for accommodating a subject to be examined, a cladding of the tubular interior space facing toward the subject to be examined and a device for lighting the tubular interior space which comprises at least one lighting element.

### BACKGROUND

In modern medicine, great value is placed on the comfort of the patient. In the case of devices having a tubular interior space into which the patient is introduced, for example computed tomography systems or magnetic resonance tomography systems, sensitive patients can experience discomfort. Many patients have a fear of confined and/or dark spaces. Hitherto, the impression of a “dark tube” could only be avoided by choosing suitable lighting for the examination space. For magnetic resonance tomography systems a system is also known in which the tubular interior space is lit by LED arrays in the lower region of the tunnel. This arrangement does however severely restrict the lighting options.

### SUMMARY

In an embodiment of the invention, a medical device is disclosed including more flexible lighting options.

The dependent claims are supported by the independent claims in this case.

A medical device is disclosed in an embodiment of the invention, wherein a lamp is mounted on the side of the cladding of the tubular interior space facing toward the subject to be examined and forms the tunnel contour of the tubular interior space.

### BRIEF DESCRIPTION OF THE DRAWINGS

Further advantages and details of the invention will emerge from the example embodiments described in the following and with reference to the drawings. In the drawings:

FIG. 1 shows a perspective view of an example embodiment of a medical device according to the invention,

FIG. 2 shows an isometric representation of the tubular interior space of a medical device according to an example embodiment of the invention designed as a computed tomography system,

FIG. 3 shows a sectional view of the tubular interior space from FIG. 2,

FIG. 4 shows an isometric representation of a computed tomography system tunnel of a further example embodiment of a medical device according to the invention,

FIG. 5 shows a longitudinal sectional view of the computed tomography system tunnel from FIG. 4, and

FIG. 6 shows a sectional view of a computed tomography system tunnel of a further example embodiment of a device according to the invention.

### DETAILED DESCRIPTION OF THE EXAMPLE EMBODIMENTS

The medical device according to an embodiment of the invention is based on the idea that the tubular interior space is lit. Modern lamps have a very flat form. It is therefore possible to mount them on the inner surface of the tunnel without causing any substantial reduction in the tunnel diameter. Glass fiber mats or also films with organic light-emitting diodes can be used for example as such thin light sources. If for example a flexible light source is used, then this can be adhesively affixed or otherwise attached, following the contour of the cladding.

In order to maintain hygiene and to facilitate cleaning, the lamp or lamps are preferably arranged as a closed surface, which means that abutting edges can be avoided. In addition, the lamps can also be covered with a transparent protective coating.

Lighting the tubular interior space of a medical device offers many advantages. Thus, for example, it relieves the patient of the fear of a dark tube. It is moreover possible to distract the patient from any fears he may have by lighting the device when the patient enters the treatment room. It is however also possible to operate the device without lighting. Depending on the embodiment of the lighting, when switched off the lamps appear milky or they are invisible. When the lighting is switched off, the medical device according to the invention thus fits well into its surroundings. The lighting can then be switched on at any time when required.

Apart from affixing the lamps in the interior of the tunnel, no further modifications of the medical device are necessary. It is therefore easily possible to upgrade existing devices with such lighting equipment. It is advantageous if the lamp comprises a plurality of lighting elements. As a result of the use of a plurality of lighting elements it is for example possible to ensure a more even illumination, but it is also possible to distribute the illumination in such a manner in the tube as to avoid dazzling the patient, given an overall high level of brightness. If a plurality of lighting elements are present, then it can also be possible to control said lighting elements independently. In this case the lighting can be matched to the wishes of a patient.

It is possible that the lamp comprises at least one mat, which is woven from light-conducting fibers, into which the light from at least one light source is coupled. The transmission of light in the light guide is effected by way of reflection at the boundary surfaces, either by way of a difference in the refractive indices or by way of a reflective layer. With regard to the mats used here, which are woven from light-conducting fibers, an even coupling-out of the light takes place over the entire mat.

Such a light-conducting mat can be affixed to the cladding of the tubular interior space of the medical device according to an embodiment of the invention. When light from a light source is coupled into said mat, then the interior space will be evenly lit. It is also possible to affix a plurality of mats, which are woven from light-conducting fibers, to the cladding of the tubular interior space. It is then possible to couple the light from a plurality of independently driven light sources into the various mats, which are woven from light-conducting fibers. The brightness or the color of individual mats can thus be regulated.

It is possible that the light-conducting fibers of the medical device according to an embodiment of the invention are designed as glass fibers and/or synthetic fibers. It is moreover advantageous if the mat, which is woven from light-conducting fibers, is flexible such that the mat can be introduced particularly simply into the tunnel and affixed there. Furthermore, a tunnel contour can be formed which corresponds more accurately to the contour of the cladding onto which the mat is affixed. This means that very little volume is occupied by the lighting.

It is advantageous if the light source is arranged outside the tubular interior space. This means that it is above all possible to avoid introducing additional electrical components into the medically relevant region of the device. This is advantageous because it means that image interference can be avoided. Furthermore, hard radiation or high magnetic fields are employed in many medical devices. Both can be problematic for the functioning and/or the service life of electrical components. It is possible to integrate the light source in the device. If the light source is mounted in an easily accessible region of the device, then it is also easily possible to replace the light source.

In order to achieve lighting which is as energy-efficient as possible and to minimize any additional heating of the medical device it can be advantageous if a light-reflecting layer is applied on the side—facing away from the subject to be examined—of the mat, which is woven from light-conducting fibers. In the case of many mats which are woven from light-conducting fibers, the light is coupled out evenly on both sides of the mat. The device cladding has a matt finish in many cases. A large part of the light on the side facing away from the patient is therefore absorbed by the device cladding and thus heats the device instead of lighting the tunnel. This is prevented by way of an additional reflective layer. It is also possible that a coating on the side—facing towards the subject to be examined—of the mat, which is woven from light-conducting fibers, prevents or reduces the coupling-out of light at at least one part of the surface. It can for example be desirable that somewhat less light is coupled out in the direct field of view of the patient in order to avoid a dazzling effect. On the other hand it can be desirable to accentuate certain patterns. Furthermore, such a coating can also be utilized in order to specifically select regions in which the tubular interior space is lit. Specific lighting in strips or other lighting areas for example is then possible.

It is also possible that at least one lamp comprises at least one LED tile having a plurality of LEDs arranged on a substrate. LED tiles comprise a generally flat substrate, on which are arranged a plurality of LEDs, and also a protective coating which as a rule is hard and water-repellent. Through the choice of the size of the LED tiles it is possible either to light larger flat areas or, when using smaller tiles, to obtain a tunnel contour which is virtually tubular. The use of LED tiles compared with individual LEDs has the principal advantage of easier wiring. Thus it is possible for all the LEDs on one LED tile to be controlled by way of a common control line. It is however also possible that LEDs of different colors can be controlled separately. Furthermore, it is possible that the LED tile has an integrated control device. In this case, it is for example possible to drive the LED tile by way of a digital control bus.

It is also possible that signals are forwarded between different LED tiles. This can then be done particularly simply if control of the LED tile is effected digitally. In this case the control signal can simply be forwarded. It is also possible to use a control protocol in which specific addressing of an individual tile is possible, even if the same digital control signal is directed to all the tiles. It is especially possible here that the control device is connected only directly to one tile which is in turn connected to its neighbors. If this is continued in this fashion, then it is possible to control all the tiles with minimal wiring. Alternatively, it is however also possible to affix contact strips onto the cladding of the tubular interior space, or a backing with contact strips can be used. It is thereby possible for example to drive the LED tiles by way of a passive matrix.

When LED tiles are used a gap is normally present between adjacent tiles. Since this situation should be avoided for reasons of hygiene it is advantageous to cover the tiles with a protective coating. It is possible that the LED tile is designed to deliver diffuse light. This for example is possible in that the protective coating, which is arranged in front of the LEDs, is roughened or contains diffusing particles. Fine imprints are also possible. Diffuse lighting is in many cases perceived as more pleasant than punctiform lighting as is produced by individual LEDs.

It is particularly advantageous if the brightness and/or color of the LEDs can be controlled independently. It is thereby possible to present a multiplicity of patterns and color profiles. It is also possible for the lighting to be matched to the wishes of the patient. Furthermore, the lighting can also be utilized in order to give information to the patient. For example, a change in a color, or a pattern formed by the lighting can indicate to the patient that he should hold his breath.

It is also possible that at least one lamp of the medical device according to an embodiment of the invention comprises at least one organic light-emitting diode (OLED), preferably at least two organic light-emitting diodes, which is/are arranged on a carrier substrate. The use of organic light-emitting diodes for lighting the tubular interior space offers numerous advantages. The power consumption of organic LEDs is lower than that of semiconductor LEDs. Higher luminous densities can thereby be achieved without a cooling facility being required. Furthermore, it is possible to fit OLEDs on larger areas and thereby to achieve a very high density of OLEDs. High-resolution, controllable lighting is thus possible. This can be utilized for example in order to display images or texts. OLEDs are in part capable of being printed. This means that methods which are related to conventional inkjet printing can be employed in order to apply OLEDs onto a substrate. The manufacture can thus be very cost-effective. It is also possible to apply OLEDs onto flexible substrates. This has the advantage that the contour of the tubular interior space can be matched very well to the cladding of the tubular interior space. Little volume is thus required and the space for the patient is less restricted. OLEDs are obtainable in different colors and also as white-light OLEDs.

It is advantageous if the at least two organic light-emitting diodes are designed to emit light of at least two different colors, in which case the brightness of the at least two organic light-emitting diodes can preferably be controlled independently. A capability to control the OLEDs independently has the major advantage that any desired patterns can be displayed. Control by way of a passive matrix is thus possible for example, in other words the OLEDs are situated at line crossings, or by way of an active matrix with the aid of thin-film transistors. In order to light the large tunnel area with OLEDs located closely together it is advantageous if individual sections of the lighting are equipped with their own control logic. It is particularly advantageous if the control logic is situated on the same substrate as the OLEDs. In this case the individual OLEDs can be driven digitally. Control signals can thus be transmitted in succession by way of a commonly used line. This considerably reduces the wiring requirement.

It is particularly advantageous if the at least two light-emitting diodes are arranged as a group and a plurality of said groups are arranged on a carrier substrate. Groups of the same kind are preferably repeated pseudoperiodically on the substrate. For example, a repetition in a rectangular grid is possible. If for example each of the groups consists of a red, a green and a blue organic light-emitting diode, each of said groups can be understood to be an RGB LED or a pixel. When observed from a sufficient distance, said organic light-emitting diodes can no longer be resolved and appear as a point having a mixed color. It is thereby possible to display full-color patterns, images or videos.

It is particularly advantageous if the carrier substrate is flexible. It is thereby possible to introduce easily into the tubular interior space, it is particularly easy to affix and the space occupied by lighting equipment is minimized. It is however also possible that the OLEDs themselves are arranged on a flexible carrier substrate but they are driven by a logic circuit which is arranged on a hard substrate. Since the drive logic is considerably less extensive, very good matching to the tubular contour of the interior space is nevertheless possible. It is particularly advantageous if the medical device according to the invention has a control unit which is designed to switch one or more of the lighting elements on or off in time-dependent fashion and/or by way of external control and/or to define the color of one or more lighting elements.

The design of the control device is dependent on the specification of the lamp to be driven. If glass fiber mats adhesively affixed into the tunnel are used for example, then the control unit controls at least one light source, the light from which is coupled into at least one of said mats. It can be possible for example that the light from a single light source is coupled into all the glass fiber mats, in which case the color of said light source is however controllable. In this case the control facility can stipulate red, green and blue components of the light. In other words, also the color and the brightness of the lighting can be determined. It is however also possible to affix a plurality of glass fiber mats to the cladding of the tubular interior space, into each of which mats is coupled the light from a separate light source. In this case the control unit can for example stipulate light color and brightness of all said control units. It is thereby possible to display patterns or color profiles.

When using OLEDs or LED tiles, each LED or OLED can for example be driven individually, thereby enabling maximum flexibility of the lighting, in which case the drive can be effected digitally. Direct drive control of a plurality of LEDs or OLEDs on the one hand is very complex.

Alternatively, it is however also possible to drive the LEDs or OLEDs in groups. It is possible to separately drive a front, central and rear tunnel section. To this end, for example the colors red, green and blue can be driven with analog signals for the front, central and rear sections of the tubular interior space. Very many other possibilities are naturally also conceivable.

By way of such a control facility, it is possible to tailor the illumination to the wishes of a patient, for example to set the color to the favorite color of a patient or to reduce the brightness. It is particularly advantageous if the control unit is designed in order to display color profiles and/or patterns by way of the lighting elements. This can be used for a plurality of functions, for example in order to distract or provide information for the patient. A slowly varying play of colors can for example keep the attention of the patient and thereby reduce the time of the examination for him. It is however also possible to utilize the lighting in order to give the patient signals. For example, the patient can be told beforehand that a change in the lighting color to red means that he should hold his breath.

The programming can take place for example directly on the device or the lighting of the device can be controlled externally. In many cases it is possible that the color profiles and/or patterns can be changed by the control unit while the medical device is in use. This enables for example dynamic displays of colors for patient information purposes or the display of changing patterns.

A medical device can be produced with control units of differing complexity. For example, exclusively static lighting can be possible in a simple embodiment, but dynamic lighting or color profiles can be possible in a more complex embodiment. The controller can moreover be designed so as to be easily replaceable. The medical device according to an embodiment of the invention can thereby be easily matched to the demands of the customer.

It is moreover possible that the color profiles and/or patterns are associated with at least one state variable of the medical device, in particular with the timing profile of an examination or treatment and/or a measurement or treatment method. Thus for example, the progress of an examination can be displayed even with single-color lighting. For example, the lighting can switch from red through yellow to green during the course of the examination. Such a switch is possible either between discrete lighting colors or continuously. It is also possible that the progress of the treatment for example is displayed by way of a strong lighting migrating from left to right or vice versa. Furthermore, colors of the lighting can for example be associated with certain examinations.

If the individual lighting elements are arranged sufficiently densely, then for example texts, images or videos can also be displayed. The individual lighting elements serve as pixels here. It is possible to display texts by switching on individual lighting elements or by switching off individual lighting elements. Information and/or instructions can thereby be given to the patient. This can also take place with the aid of pictograms. It is also possible to display images or videos, delivered both from an internal memory, directly from a storage medium or by way of an external video interface. It is also possible for example in the event of patient movements to also move the image with the patient. This serves principally to entertain the patient and thereby to allay fears. The facility can however also be used to provide the patient with information. It is also possible to display the images, texts or videos displayed by the lighting equipment such that they are also moved in the event of movement of the patient by way of a patient positioning device.

The medical device according to an embodiment of the invention can be designed for example as a computed tomography system or as a magnetic resonance tomography system.

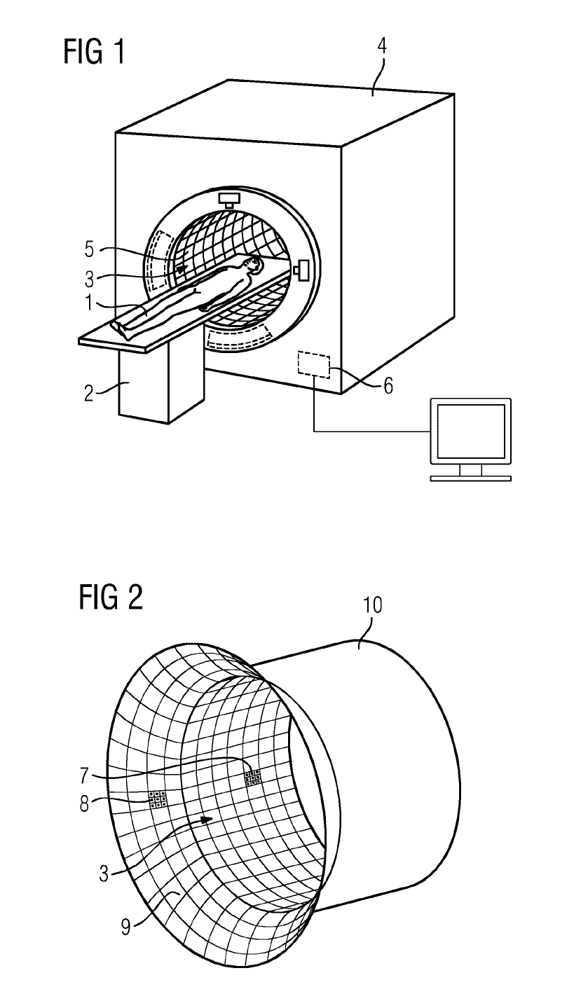

FIG. 1 shows a perspective view of an example embodiment of a medical device. The subject to be examined 1, a patient, is introduced by way of a patient positioning device 2 in the form of a patient examination table into a tubular interior space 3 of a medical device 4. The tubular interior space 3 is lit by way of lamps 5. Control of the lighting is effected by way of a control unit 6.

The lamps 5 are designed as LED tiles. Through the use of small tiles it is possible to form a tubular contour and thereby to obtain a maximum free volume in the tubular interior space 3. Each of the LED tiles contains a plurality of separately drivable RGB LEDs, thereby enabling lighting of the tubular interior space with patterns and in different colors. The individual LED tiles are driven digitally and through a matrix composite between the tiles. Each individual LED tile contains a controller for the individual LEDs incorporated. Said control unit can be addressed by way of commands which are transferred by way of a serial digital interface. The transfer of the control signals from the control unit 6 to the LED tiles takes place by way of networking between the tiles. In this case digital control signals are forwarded between adjacent tiles. Addressing of an individual LED tile can take place by assigning the LED tile an identification string which is transferred with each control command.

The LED tiles are affixed to the cladding of the tubular interior space 3 by an adhesive. After being adhesively affixed, the tiles are additionally covered with a thin layer of a transparent protective coating. This means that the cladding of the tubular interior space 3 is smooth and can be easily cleaned. This is important particularly in respect of hygiene requirements.

FIG. 2 shows an isometric representation of an example embodiment designed as a computed tomography system tunnel, in which LED tiles 7, 8 are used as the lighting element. LED tiles 7 are mounted in the tubular interior space 3 and LED tiles 8 are also arranged on the funnel 9 which is arranged at the outer open end of the tubular interior space 3. The arrangement of the LED tiles 7, 8 is only illustrated by way of example in each case at two points. The LED tiles 7, 8 are affixed to the cladding 10 by an adhesive. The LED tiles 7, 8 are covered by a thin transparent protective coating (not shown).

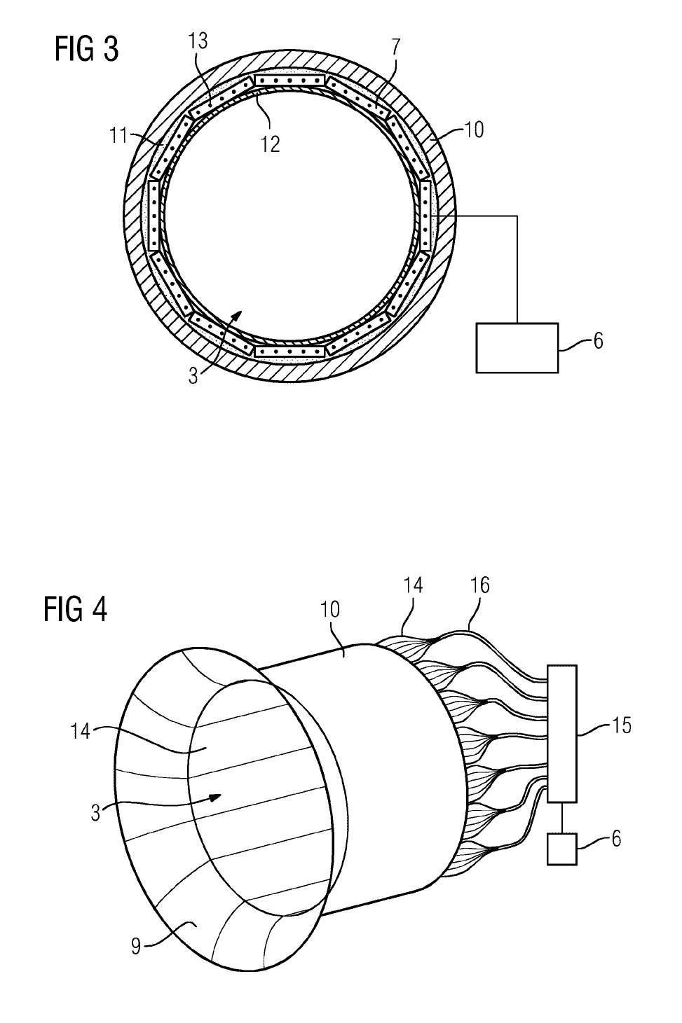

FIG. 3 is a sectional view of the computed tomography system tunnel shown in FIG. 2 and shows the arrangement of the LED tiles 7 on the cladding 10 of the tubular interior space 3. The LED tiles 7 are affixed on the cladding 10 with the aid of an adhesive layer 11. The LED tiles used are rectangular and therefore there are edges between the LED tiles at which dirt could accumulate. An additional transparent protective coating 12 is therefore applied to the LED tiles 7, thereby forming a smooth, easily cleaned surface. The individual LED tiles 7 consist of a plurality of RGB LEDs 13. Each individual one of said RGB LEDs can be controlled individually in respect of color and brightness. The control is effected digitally. To this end, a control circuit (not shown) is arranged on each of the LED tiles. Said control circuit receives commands from the control facility 6. Such a command consists of an identification string which enables the addressing of individual LED tiles 7, and also of a range of light intensities for the individual colors of the individual RGB LEDs.

Such a system can enable individual LEDs to be controlled with a minimal wiring because digital control commands can be forwarded from one tile to the adjacent tiles. Thus only a connection of the control facility 6 to an LED tile 7 and a connection of the LED tiles 7 to one another are required.

FIG. 4 is an isometric representation of a computed tomography system tunnel of a further example embodiment. Here the lighting of the tubular interior space 3 is implemented by way of glass fiber mats 14 into which the light from a light source 15 is coupled. The glass fiber mats are designed in strips, wherein the strips are arranged in rectangular fashion along the length of the tunnel and are somewhat wider on the side of the funnel 9 in order to enable complete coverage of the funnel 9 with glass fiber mats.

A plurality of layers are arranged on the cladding 10. Directly on the cladding 10 is arranged a reflective layer 17, which is designed as a reflective coating, on which are arranged the glass fiber mats 14. The glass fiber mats 14 are affixed by impregnating the glass fiber mats 14 with epoxy resin. The epoxy resin has several functions. Firstly, it serves as an adhesive layer 11 for affixing the glass fiber mats 14 on the cladding. Furthermore, after the epoxy resin layer has cured the glass fiber mats 14 are hard. This means that the glass fiber mats 14 are stabilized in the tubular interior space 3. If a somewhat greater quantity of epoxy resin is used, then this itself also directly forms the protective coating 12. An additional protective coating 12 over the glass fiber mats 14 is important because otherwise abutting edges and joints can be produced at the transitions between the glass fiber mats 14, at which deposits of dirt may accumulate. This situation is avoided by the protective coating 12. The individual glass fibers of the glass fiber mat 14 are led out at the side of the tunnel facing away from the funnel and combined to form glass fiber bundles 16. Each glass fiber mat 14 is assigned a separate glass fiber bundle 16. The glass fiber bundles 16 are connected to the light source 15. The light sources can be controlled individually for each of the glass fiber bundles 16 in respect of both brightness and also color. The selection of color and brightness is effected by the control facility 6.

The tubular interior space 3 can be lit stripwise in different colors and brightnesses. It is possible to light parts of the glass fiber mats 14 in a single color, in the favorite color of the patient, during a medical intervention, while individual glass fibers are used in order to display the progress of treatment. This can be done by way of a continuous change in color from red through yellow to green. Alternatively, it is also possible to move the lighting from left to right or vice versa in the tube during the treatment. It is also possible to give the patient instructions by changing the color of the all or individual glass fiber mats, for example to hold his breath.

FIG. 5 shows a longitudinal sectional view of the computed tomography system tunnel from FIG. 4. The layered structure of the lighting equipment can be seen here. The cladding 10 of the tubular interior space 3 is firstly provided with a reflective layer. The glass fiber mats 14 are adhesively affixed onto said layer. A protective coating 12 is applied onto the glass fiber mats 14. This serves to enable improved cleaning of the medical device. The glass fiber mats run along the tubular interior space 3 and end at the funnel 9. The same also applies to the reflective layer 17 and the protective coating 12. Likewise shown is the connection between the glass fiber bundle 16 and the light source 15 which is driven by the control unit 6.

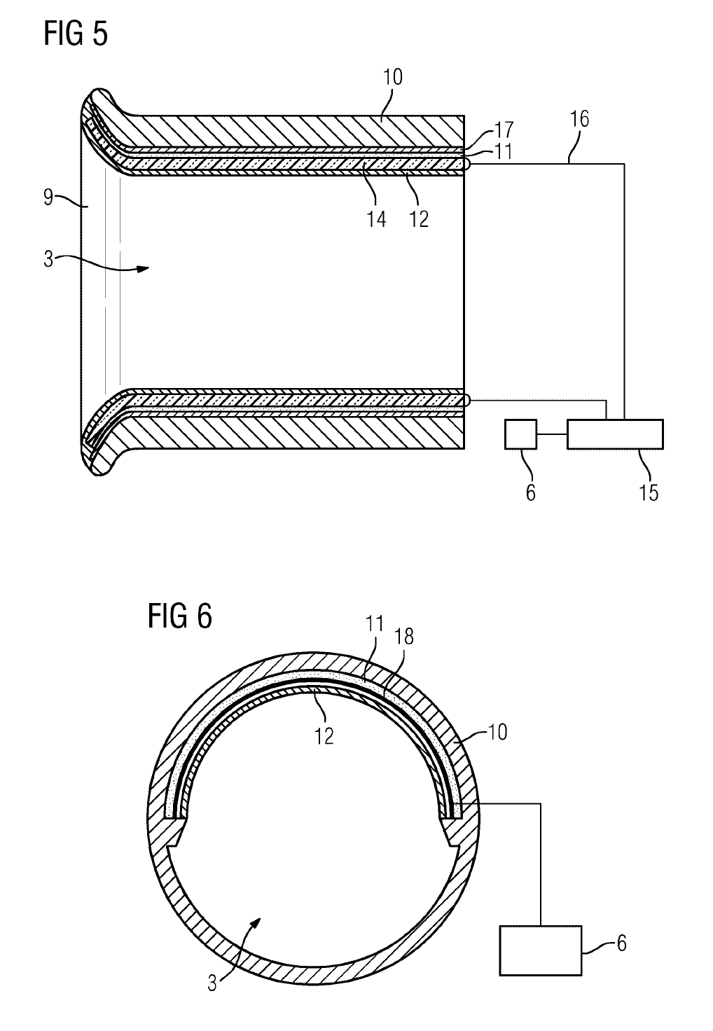

FIG. 6 shows a sectional view of a computed tomography system tunnel of a further example embodiment. Here the lighting of the tubular interior space 3 is implemented by way of an OLED film 18. The OLED film 18 is affixed to the cladding 10 of the tubular interior space 3 by way of an adhesive layer 11. Only the upper half of the cladding 10 is covered with OLED film 18. The lower half of the cladding 10 lies outside the field of view of the patient and does not therefore need to be lit.

The OLED film 18 is additionally covered with a protective coating 12 which enables better cleaning of the medical device. In addition, OLED films 18, particularly those on flexible substrates, may be susceptible to mechanical damage. A drive logic circuit which is designed as an active matrix is likewise incorporated into the OLED film. The OLED film can be driven digitally. It is thereby possible to display temporally varying patterns and plays of color. It is also possible to display images or videos. If the patient is moved inside the tube during the course of the treatment, then the image or video display can also be moved accordingly.

Although the invention has been illustrated and described in detail by way of the preferred example embodiment, the invention is not restricted by the disclosed examples and other variations can be derived therefrom by the person skilled in the art without departing from the scope of protection of the invention.

1. A medical device, comprising:
a tubular interior space to accommodate a subject to be examined;
a cladding of the tubular interior space, facing the subject to be examined; and
a device to light the tubular interior space, the device including at least one lamp, the at least one lamp being mounted on a side of the cladding of the tubular interior space facing towards the subject to be examined and forming a tunnel contour of the tubular interior space, the at least one lamp including
at least one flexible mat woven from light-conducting fibers, into which the light from at least one light source is coupled, or
at least two organic light-emitting diodes, arranged on a flexible carrier substrate.

2. The medical device of claim 1, wherein the at least one lamp includes a plurality of lighting elements.

3. The medical device of claim 1, wherein the light-conducting fibers are designed as at least one of glass fibers and synthetic fibers.

4. The medical device of claim 1, wherein the light source is arranged outside the tubular interior space.

5. The medical device of claim 1, wherein a light-reflecting layer is applied on the side facing away from the subject to be examined of the mat which is woven from light-conducting fibers.

6. The medical device of claim 1, wherein a coating on the side facing towards the subject to be examined of the mat, which is woven from light-conducting fibers, prevents or reduces the coupling-out of light at at least one part of the surface.

7. The medical device as claimed in claim 1, wherein the at least two organic light-emitting diodes are designed to emit light of at least two different colors.

8. The medical device of claim 1, wherein the at least two light-emitting diodes are arranged as a group and a plurality of said groups are arranged on a carrier substrate.

9. The medical device of claim 1, wherein the medical device includes a control unit, designed to at least one of
switch one or more of the lighting elements on or off in at least one of a time-dependent fashion and by way of external control, and
define the color of one or more lighting elements.

10. The medical device of claim 9, wherein the control unit is designed to display at least one of color profiles and patterns by way of the lighting elements.

11. The medical device of claim 10, wherein the at least one of color profiles and patterns are associated with at least one state variable of the medical device.

12. The medical device of claim 1, wherein the medical device is designed as a computed tomography system or as a magnetic resonance tomography system.

13.-19. (canceled)

20. The medical device of claim 2, wherein the light-conducting fibers are designed as at least one of glass fibers and synthetic fibers.

21. The medical device as claimed in claim 7, wherein the brightness of the at least two organic light-emitting diodes is independently controllable.

22. The medical device of claim 2, wherein the at least two light-emitting diodes are arranged as a group and a plurality of said groups are arranged on a carrier substrate.

23. The medical device of claim 10, wherein the at least one of color profiles and patterns are changeable by the control unit while the medical device is in use.

24. The medical device of claim 11, wherein the at least one of color profiles and patterns are associated with at least one of the timing profile of an examination or treatment and a measurement or treatment method.
